(19) United States
(12) Patent Application Publication
(10) Pub. No.: US 2025/0032759 A1
(43) Pub. Date: Jan. 30, 2025

(54) GUIDEWIRE INCLUDING A CORE WIRE LOCKING ELEMENT

(71) Applicant: Covidien LP, Mansfield, MA (US)

(72) Inventors: Daniel J. Deen, Long Beach, CA (US); Ashok R. Nageswaran, Irvine, CA (US); Tejashri Kumar, Fountain Valley, CA (US)

(21) Appl. No.: 18/784,073

(22) Filed: Jul. 25, 2024

Related U.S. Application Data
(60) Provisional application No. 63/515,704, filed on Jul. 26, 2023.

(51) Int. Cl. A61M 25/09

(52) U.S. Cl. CPC A61M 25/09; A61M 2025/09075; A61M 2025/09108; A61M 2025/09175; A61M 2205/02; A61M 2207/10

(57) ABSTRACT

A guidewire comprising: a core wire; an elongated member extending along a longitudinal axis and defining: an inner surface defining an inner lumen, a plurality of openings disposed along the elongated member, each opening extending from an outer surface of the elongated member towards the inner surface, a first locking recess extending through the elongated member from the outer surface to the inner surface, a second locking recess extending from the inner surface towards a second location on the outer surface, wherein the first locking recess and the second locking recess defines a locking channel; and a locking element disposed within the locking channel and affixed to the elongated member, the locking element defining an aperture configured to receive the core wire, wherein the locking element is configured to inhibit unintended proximal movement of the core wire along the longitudinal axis relative to the elongated member.

### CROSS-REFERENCE TO RELATED APPLICATIONS

This application claims the benefit of and priority to U.S. Provisional Patent Application Ser. No. 63/515,704 filed Jul. 26, 2023, the entire disclosure of which is incorporated by reference herein.

### TECHNICAL FIELD

This disclosure relates to a medical guidewire.

### BACKGROUND

A medical catheter defining at least one lumen has been proposed for use with various medical procedures. For example, in some cases, a medical catheter may be used to access and treat defects in blood vessels, such as, but not limited to, lesions or occlusions in blood vessels. A medical guidewire may be disposed within the catheter lumen and configured to control navigation of the medical catheter within the body of a patient.

### SUMMARY

In some examples, a guidewire includes a core wire and an elongated member including a proximal section and a distal section. The distal section includes a wall defining one or more openings that extend at least partially through a thickness of the wall of the elongated member. The guidewire may also include one or more other elements, such as, but not limited to, an outer jacket or a support member (e.g., a coil and/or a braid). The one or more openings defined in the wall of the distal section of the elongated member may increase a bending flexibility of the distal section relative to the proximal section of the elongated member without reducing a tensile strength of the elongated member below a threshold for navigability through the vasculature.

This disclosure describes example locking elements configured to be disposed within a guidewire and couple the core wire of the guidewire to the elongated member. The locking element may be affixed to one or both of the core wire or the elongated member and configured to inhibit unintended movement of the core wire relative to the elongated member, or vice versa. In response to fracture of the core wire and/or elongated member, the locking element may continue to secure fractured portions of the core wire and/or elongated member to the guidewire, such that the fractured portions may be removed. This disclosure also describes examples of methods of forming a guidewire with a locking element and examples of methods of manufacturing the example locking elements as described herein.

In some examples, this disclosure describes a guidewire comprising: a core wire defining a longitudinal axis; an elongated member extending along the longitudinal axis, the elongated member defining: an inner surface defining an inner lumen, wherein a distal section of the core wire is positioned within the inner lumen, a plurality of openings disposed along a portion of the elongated member, each opening of the plurality of openings extending from an outer surface of the elongated member towards the inner surface of the elongated member, a first locking recess distal to the plurality of openings, the first locking recess extending through the elongated member from a first location on the outer surface of the elongated member to the inner lumen, a second locking recess distal to the plurality of openings, the second locking recess extending from the inner surface of the elongated member towards a second location on the outer surface of the elongated member, wherein the second location is different from the first location, and wherein the first locking recess and the second locking recess defines a locking channel extending from the first location; and a locking element disposed within the locking channel and the inner lumen and affixed to the elongated member, the locking element defining an aperture, wherein a distal end of the core wire extends through the aperture, wherein the locking element is configured to inhibit unintended proximal movement of the core wire along the longitudinal axis relative to the elongated member.

In some examples, this disclosure describes a method of manufacturing a guidewire of claim 1, the method comprising: inserting the locking element into the locking channel on the elongated member via the first locking recess; inserting a distal end of the core wire through the inner lumen of the elongated member and the aperture of the locking element; affixing the distal end of the core wire to the locking element, wherein when the distal end of the core wire is affixed to the locking element, the locking element inhibits unintended proximal movement of the distal end of the core wire along the longitudinal axis relative to the elongated member; and forming a distal tip of the guidewire around the distal end of the core wire and a distal end of the elongated member, wherein the distal tip of the guidewire is configured to inhibit unintended movement of the core wire relative to the elongated member.

In some examples, this disclosure describes a locking element configured to be inserted into a locking channel of a guidewire, the locking element comprising: a central body extending along a first axis from a proximal surface to a distal surface and from a first end to a second end along a second axis, wherein the second axis is orthogonal to the first axis; one or more extensions extending away from the second end of the central body and away from the second axis, wherein each extension of the one or more extensions extend along the first axis from one of the distal surface or the proximal surface towards the other of the distal surface or the proximal surface; and an aperture extending along the first axis from the proximal surface to the distal surface, wherein the aperture is configured to receive a core wire of the guidewire when the locking element is inserted into the locking channel within an elongated body of the guidewire, and wherein the distal surface is configured to interface with the core wire to inhibit unintended proximal movement of the core wire along the first axis.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

FIG. 1 is a conceptual diagram illustrating example guidewire having an elongated member.

FIG. 2A is a conceptual diagram illustrating an elongated member of the example guidewire of FIG. 1 with an example locking element.

FIG. 2B is a conceptual diagram illustrating a cross-sectional view of the distal section of the example guidewire of FIG. 2A, the cross-section being taken along line A-A in FIG. 2A.

FIG. 2C is a conceptual diagram illustrating another example cross-sectional view of the distal section of the example guidewire of FIG. 2A, the cross-section being taken along line B-B in FIG. 2A.

FIG. 3A is a conceptual diagram illustrating a front view of an example locking element.

FIG. 3B is a conceptual diagram illustrating a side view of a first end of the example locking element of FIG. 3A.

FIG. 3C is a conceptual diagram illustrating a side view of a second end of the example locking element of FIG. 3A.

FIG. 3D is a conceptual diagram illustrating a top view of the example locking element of FIG. 3A.

FIG. 4 is a conceptual diagram illustrating another example elongated member of the example guidewire of FIG. 1 with a plurality of example locking elements.

FIG. 5A is a conceptual diagram illustrating another example elongated member of the example guidewire of FIG. 1 with a plurality of example locking elements.

FIG. 5B is a conceptual diagram illustrating a cross-sectional view of the distal section of the example guidewire of FIG. 5A, the cross-section being taken along line C-C in FIG. 5A.

FIG. 6 is a flow diagram illustrating an example method of manufacturing the guidewire of FIG. 1.

### DETAILED DESCRIPTION

Medical devices described herein may be used with a medical catheter (“catheter”) that includes a relatively flexible catheter body configured to be navigated through vasculature of a patient, e.g., tortuous vasculature in a brain of the patient. The catheter may be navigated within the vasculature via use of a medical guidewire (“guidewire”). The guidewire includes a relatively flexible distal section that may exhibit increased flexibility relative to a proximal section of the guidewire.

In some examples, a guidewire described herein includes an elongated member (also referred to herein as “elongated tube”), a support element (e.g., a coil member or a braided member, or combinations thereof), a core member (e.g., a core wire) and an outer jacket, which can interact to provide a relatively flexible elongated member with sufficient structural integrity (e.g., column strength, which may be a measure of a maximum compressive load that can be applied to the elongated member without taking a permanent set) to permit the guidewire to be advanced through the vasculature via a pushing force applied to a proximal section of the guidewire, e.g. without buckling, kinking, or otherwise undesirably deforming (e.g., ovalization). A distal section of the guidewire may lead the guidewire through vasculature of a patient.

The increased flexibility of the distal section may be at least partially (e.g., partially or fully) attributable to the configuration of the elongated member. For example, a distal section of the elongated member may include one or more openings (also referred to herein as “voids” and/or “cuts”), which help to increase a bending flexibility of the distal section of the elongated member while maintaining a desirable tensile strength of the elongated member. The one or more openings may have any suitable configuration that helps to increase the bending flexibility of the elongated member while maintaining tensile strength of the elongated member and the overall guidewire. Each of the one or more openings may be an absence of material in or a locally thinner portion of the wall (e.g., a groove, divot, pocket, through-hole, or the like in an otherwise continuous surface), or may be an incision in the wall of the elongated member that is formed without removing material from the wall. While the examples of this disclosure primarily describe the elongated member and the openings on the elongated member with reference to a guidewire, an elongated member as described in any of the examples included herein may be used in other medical applications, e.g., as an inner liner or a support member in a medical catheter, or the like.

The elongated member may increase the flexibility of the distal section of the guidewire and therefore increase the navigability of the guidewire through vasculature compared to a guidewire including an elongated member that is otherwise the same, but does not include one or more openings in a distal section. The elongated member may be formed from a biocompatible metallic alloy, e.g., Nitinol. The metallic alloy may be formed into an elongated tube (e.g., a hypotube or the like) defining the elongated member. In some examples, the elongated member may be formed from one or more polymers (e.g., Polytetrafluoroethylene (PTFE) or the like) into an elongated tube.

In some examples, the flexible guidewire is configured to substantially conform to the curvature of the vasculature. In addition, in some examples, the guidewire may define a column strength and flexibility that allow at least a distal section of the guidewire to be navigated from a femoral artery, through the aorta of the patient, and into the intracranial vascular system of the patient, e.g., to reach a relatively distal treatment site, including the middle cerebral artery (MCA), internal carotid artery (ICA), the Circle of Willis, and tissue sites more distal than the MCA, ICA, and the Circle of Willis. The MCA and, consequently, vasculature distal to the MCA may be relatively difficult to access due to the carotid siphon anatomy that must be traversed to reach such locations.

Although primarily described as being used to reach relatively distal vasculature sites, the guidewires described herein may readily be configured to be used with other target tissue sites. For example, the guidewire may be used to access tissue sites throughout the coronary and peripheral vasculature, the gastrointestinal tract, the urethra, ureters, Fallopian tubes, and other body lumens.

Prior to using a guidewire, a clinician may shape the guidewire into a desired shape. In some examples, the clinician may bend or wrap the guidewire (e.g., around a mandrel, a guidewire shaping tool) and apply a tensile force to the guidewire until the guidewire forms a desired shape. Different desired shapes may correspond to the guidewire defining different bending angles, different shapes, different radii of curvature, or the like. The clinician may shape the guidewire into different shapes based on the shape and/or location of a body lumen the clinician intends to introduce the guidewire into.

During the shaping of the guidewire by the clinician, the forces applied on the guidewire may lead to one or more unintended effects including, but are not limited to, unintended stretching of the elongated member, fracture at one or more locations along the elongated member, separation of a distal tip of the guidewire from the core wire and/or the elongated member, or unintended stretching of a coil within the elongated member. The unintended effects may lead to separation of components of the guidewire while the guidewire is within the body of the patient, resulting in unintended interactions between the separated components of the guidewire and the tissue of the patient.

In some guidewires, the distal tip may secure various components of the guidewire together. For example, the distal tip may be affixed to both the core wire and the elongated member of the guidewire and may inhibit unintended movement of the core wire relative to the elongated member, or vice versa. However, as a result of the unintended effects experienced by the guidewire, the distal tip of the guidewire may become separated from one or both of the core wire or the elongated member, thereby allowing for unintended movement between the core wire and the elongated member. In some examples, fracture at one or more locations along the elongated member may lead to partial or complete separation of portions of the elongated member. Separated portions may then be detached and separate from the remainder of the guidewire.

This disclosure describes a locking element configured to inhibit unintended movement between the core wire and the elongated member. The locking element may be affixed to both the core wire and the elongated member. An example guidewire including an example locking element described herein may provide several benefits over an identical guidewire without locking elements. In some examples, the locking element may provide additional safety measures and prevent unintended separation of components of the guidewire from the remainder of the guidewire by providing another point of fixation between the distal tip, core wire, and/or the elongated member of a guidewire. In some examples, the locking element may allow for retention of separated portions of the elongated member within the guidewire in the event of a partial or complete fracture of the elongated member, thereby reducing a likelihood of unintended effects on the patient. In some examples, the locking element may provide for increased torque transmission between the core wire and the elongated member, thereby increasing the responsiveness of the guidewire.

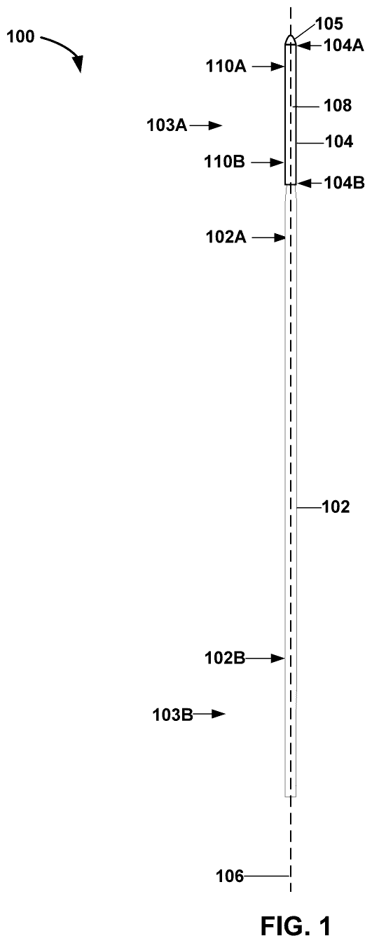

FIG. 1 is a conceptual diagram illustrating example guidewire 100. Guidewire 100 may define a longitudinal axis 106 extending between a distal section 103A and a proximal section 103B. As shown, guidewire 100 includes a core wire 102, an elongated member 104, and an distal tip 105. Core wire 102 extends along longitudinal axis 106 of guidewire 100 between a distal section 102A and a proximal section 102B. Elongated member 104 extends along longitudinal axis 106 between a distal end 104A and a proximal end 104B, and defines an inner lumen. Distal end 104A may be coupled to distal tip 105 and/or to core wire 102. A portion of core wire 102 may be disposed within the inner lumen defined by elongated member 104. Elongated member 104 may be configured to retain distal section 102A of core wire 102, such that elongated member 104 is mechanically responsive to a force exerted on proximal section 102B of core wire 102. For example, elongated member 104 may be configured to retain distal section 102A of core wire 102 within the inner lumen defined by elongated member 104. As described in greater detail below, elongated member 104 may define one or more openings in a wall of elongated member 104.

Guidewire 100 may define a suitable length for accessing a target tissue site within the patient from a vascular access point. The length may be measured along longitudinal axis 106 of guidewire 100. The target tissue site may depend on the medical procedure for which guidewire 100 and/or an accompanying catheter is used. For example, if the catheter is a distal access catheter used to access vasculature in a brain of a patient from a femoral artery access point at the groin of the patient, guidewire 100 may have a length of at least about 125 centimeters (cm) to about 135 cm, such as about 132 cm, although other lengths may be used. Distal section 103A of guidewire 100 may be disposed within the vasculature of the patient and proximal section 103B may be disposed outside of the body of the patient. A clinician may manipulate proximal section 103B of guidewire 100 to rotate, retract, and/or advance distal section 103A of guidewire 100 within the vasculature.

Guidewire 100 may be used to access relatively distal locations in a patient, such as the middle cerebral artery (“MCA”) in a brain of a patient. The MCA, as well as other vasculature in the brain or other relatively distal tissue sites (e.g., relative to the vascular access point), may be relatively difficult to reach with a catheter, due at least in part to the tortuous pathway (e.g., comprising relatively sharp twists or turns) through the vasculature to reach these tissue sites. Guidewire 100 may be structurally configured to be relatively flexible, pushable, and relatively kink- and buckle-resistant, so that it may resist buckling when a pushing force is applied to a relatively proximal section of the catheter to advance the catheter body distally through vasculature, and so that it may resist kinking when traversing around a tight turn in the vasculature. Kinking or buckling of guidewire 100 may hinder a clinician's efforts to push the catheter body distally, e.g., past a turn. Such kinking or buckling of guidewire 100 may be more likely at distal portion 103A of guidewire 100, as distal portion 103A may experience relatively larger bending forces than proximal portion 103B as both a leading portion of guidewire 100 and a distal-most portion of guidewire 100 positioned in vasculature that may become progressively more tortuous from the vascular access point to the target tissue site.

As discussed in further detail below, one structural characteristic that may contribute to at least the flexibility of guidewire 100, particularly at distal portion 103A, is flexibility of elongated member 104. The flexibility of elongated member 104 may be based at least in part on a cut pattern of the one or more openings defined in a wall 107 of distal section 103A. The one or more openings in distal section 103A of elongated member 104 may improve the navigability of guidewire 100 through vasculature of a patient relative to another guidewire including an elongated member without any openings in the wall of the elongated member. Without being limited to any particular theory, a bending force exerted on guidewire 100 may cause guidewire 100 to bend away from longitudinal axis 106. For example, the bending force may cause guidewire 100 to bend along a plane orthogonal to longitudinal axis 106. The bending force may generate a compressive force in a portion of guidewire 100 facing toward the bending direction of guidewire 100 and a tensile force in a portion of guidewire 100 facing away from the bending direction. As compared to an identical elongated member with no openings, the one or more openings on elongated member 104 may provide discontinuities for reducing the generated tensile force (e.g., voids or cuts) and/or provide spaces for reducing the generated compressive force (e.g., voids), such that elongated member 104 may provide a reduced resistance to the bending force as compared to the identical elongated member that does not include any openings.

Core wire 102 may extend from distal tip 105 to a proximal end of proximal portion 102B. During navigation of core wire 102 within vasculature of the patient, distal section 102A may be disposed within the vasculature and proximal section 102B may be outside of the body of the patient. A clinician may operate proximal section 102B to advance, retract, rotate, and/or bend guidewire 100 within the vasculature. Core wire 102 may transmit forces and/or torques applied, for example by the clinician to proximal section 102B along longitudinal axis 106 to distal tip 105. At least a portion of distal section 102A is disposed within the inner lumen defined by elongated member 104 and transmits forces and/or torques from proximal section 102B to distal tip 105. Core wire 102 may be formed from one or more biocompatible metallic alloys including, but is not limited to, stainless steel. Core wire 102 may be selected for a variety of properties, including mechanical strength sufficient to maintain integrity and transmit forces from distal section 102B to proximal section 102A, and flexibility sufficient for core wire 102 to be positioned within a more proximal portion of the pathway in the vasculature. However, the mechanical strength of core wire 102 may be substantially higher than required for forces experienced at distal portion 103A.

Elongated member 104 is configured with a higher flexibility than core wire 102 while maintaining adequate mechanical strength. Elongated member 104 defines one or more opening(s) on outer surface of elongated member 104 from distal end 104A to proximal end 104B. The opening(s) may have varied patterns, dimensions, and/or designs between distal end 104A and proximal end 104B, e.g., to increase flexibility of distal section 110A of elongated member 104 relative to proximal section 110B of elongated member 104. For example, during navigation of the catheter through relatively tortuous vasculature, distal section 103A of guidewire 100 may experience forces along axis 106 (e.g., tensile or compressive forces), forces around axis 106 (e.g., torque), and/or forces away from axis 106 (e.g., bending forces). A distal section of an identical elongated member that does not include opening(s) may provide resistance to these forces. While resistance to forces along and around axis 106 may ensure sufficient pushability of guidewire 100, resistance to forces away from axis 106 may reduce navigability of guidewire 100. The openings may be configured to improve navigability of distal section 103A relative to an identical guidewire that does not include openings by reducing resistance of elongated member 104 to forces away from axis 106 without substantially reducing resistance of elongated member 104 to forces along and/or around axis 106 beyond that for maintaining pushability of elongated member 104. For example, a flexibility of distal section 103A of elongated member 104 that includes openings may be lesser than or equal to half a flexibility of an identical elongated member that does not include openings. An identical elongated member may be a metallic (e.g., a stainless steel) round wire of equivalent dimensions that does include any openings. In some examples, distal section 110A including openings is at least as flexible as a round metallic wire without openings that defines a larger outer diameter than distal section 110A.

In some examples, elongated member 104 includes opening(s) having individual, relative, and/or collective properties configured to provide increased flexibility to elongated member 104 as compared to an identical elongated member without any openings. For example, individual properties may include properties of an individual opening, such as size or shape; relative properties may include properties of two or more openings or groups of openings relative to each other, such as pitch; and collective properties may include properties of a plurality of the openings, such as a density of the openings, and/or flexibility of distal section 110A or portion of distal section 110A provided by the openings.

Distal section 110A of elongated member 104 may define an increased density of openings relative to a more proximal section (e.g., proximal section 110B) of elongated member 104. The increase density of openings may provide several material property benefits to distal section 110A of elongated member 104. The increased radii of curvature of openings and the reduced pitch between openings may increase tensile strength and torque response of elongated member 104 while causing distal section 110A to maintain a low bending stiffness. In some examples, the reduced pitch between openings reduces a bending radius of distal section 110A and/or reduce localized strain exerted upon any of the uncut sections of distal section 110A. Distal section 110A may define openings having a combination reduced beam lengths and pitches between openings to reduce bending stiffness and increase pliability of distal section 110A.

Distal section 110A includes distal end 104A of elongated member 104, and proximal section 110B includes proximal end 104B of elongated member 104. Distal section 110A may have any suitable length. In some examples, distal section 110A is about 5% to about 50% of a total length of elongated member 104, such as about 10% to about 40%, about 5% to about 25%, or about 10% to about 25% of the total length of elongated member 104. In some examples, distal section 110A may define a length of about 5 cm to about 40 cm, such as about 5 cm to about 35 cm, or about 5 cm to about 10 cm. In some of these examples, elongated member 104 defines a total length of about 132 cm. Distal section 110A may be configured to provide a leading end for navigating through vasculature, while proximal section 110B may be configured to comply with changing curvature of the vasculature. As such, distal section 110A and proximal section 110B may be configured with different properties corresponding to the different functions of each section.

Distal tip 105 may define a distalmost portion of guidewire 100. Distal tip 105 may be configured to prevent adverse interaction between portions of guidewire 100 (e.g., distal end 104A of elongated member 104) and tissue of the patient as guidewire 100 navigates through the vasculature of the patient. The distal tip 105 may define a blunted and/or semi-spherical shape, e.g., to prevent unintended puncture of the tissue of the patient. Distal tip 105 may be formed from biocompatible materials including, but are not limited to, a biocompatible glue, a biocompatible polymer, or the like. Distal tip 105 may be coupled to (e.g., permanently affixed to) both elongated member 104 and a distal end of core wire 102. For example, a relatively radially inward portion of a proximal end of distal tip 105 is coupled to the distal tip of core wire 102 and a relatively radially outward portion of the proximal end of distal tip 105 is coupled to the distal end of elongated member 104. Coupling distal tip 105 to both core wire 102 and elongated member 104 may secure the position and orientation of elongated member 104 relative to core wire 102 and vice versa, e.g., to prevent unintended rotation and/or separation of elongated member 104 from core wire 102.

In some examples, an outer diameter of guidewire 100 may be uniform along the length of guidewire 100. In other examples, an outer diameter of guidewire 100 may taper from a first outer diameter at proximal section 102B of core wire 102 to a second outer diameter at distal section 102A of core wire 102, the second outer diameter being smaller than the first outer diameter. In some examples, the taper may be continuous along the length of guidewire 100, such that an outer surface of core wire 102 defines a smooth transition between different diameter portions. In some examples, as illustrated in FIG. 1, distal section 102A tapers to a smaller outer diameter such that when elongated member 104 is disposed over a reduced-diameter portion of distal section 102A of core wire 102, an outer diameter of elongated member 104 defines a same or smaller outer diameter as a portion of core wire 102 proximal to proximal end 104B of elongated member 104. In other examples, core wire 102 may define discrete step-downs in outer diameter to define the taper. The size of the discrete step-downs in diameter may be selected to reduce the number of edges that may catch on anatomical features within the vasculature and/or with the catheter as guidewire 100 is advanced through vasculature.

A larger diameter proximal section 102B of core wire 102 may provide better proximal support for core wire 102 than a smaller diameter proximal section 102B, which may help increase the pushability of guidewire 100. In addition, a smaller diameter distal section 102A of core wire 102 may increase the navigability of guidewire 100 through tortuous vasculature than a larger diameter distal section 102A. Thus, reducing the outer diameter of core wire 102 at distal section 102A may improve navigability of guidewire 100 through tortuous vasculature while still maintaining a relatively high level of proximal pushability as compared to an identical core wire 102 defining a larger diameter distal section 102A or defining a uniform outer diameter.

In some examples, at least a portion of an outer surface of guidewire 100 includes one or more coatings, such as, but not limited to, an anti-thrombogenic coating, which may help reduce the formation of thrombi in vitro, an anti-microbial coating, or a lubricating coating. The lubricating coating may be configured to reduce static friction or kinetic friction between guidewire 100 and tissue of the patient and/or a catheter retaining guidewire 100 as guidewire 100 is advanced through the vasculature. The lubricating coating can be, for example, a hydrophilic coating. In some examples, the entire working length of guidewire 100 (from distal tip 105 to proximal section 102B of core wire 102) is coated with the hydrophilic coating. In other examples, only a portion of the working length of guidewire 100 coated with the hydrophilic coating. This may provide a length of guidewire 100, e.g., at proximal section 102B, with which the clinician may grip guidewire 100 (e.g., core wire 102), e.g., to rotate guidewire 100, pull guidewire 100, or push guidewire 100 through vasculature.

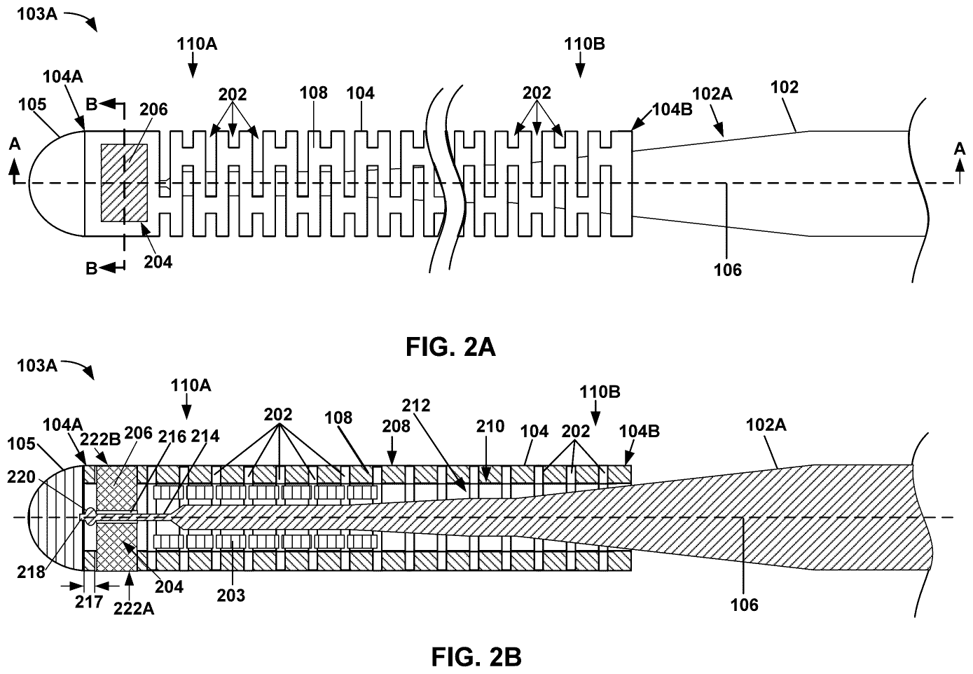

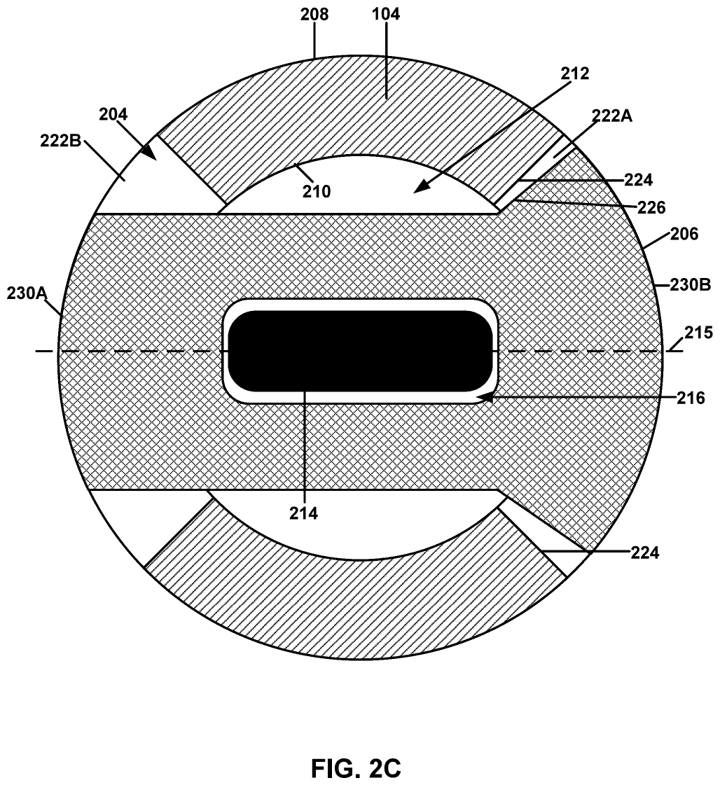

FIG. 2A is a conceptual diagram illustrating a side view of elongated member 104 of guidewire 100 of FIG. 1 with an example locking element 206. Elongated member 104 is disposed at distal section 103A of guidewire 100 and around distal section 102A of core wire 102 of guidewire 100. Elongated member 104 includes wall 108 extending along longitudinal axis 106 from distal end 104A to proximal end 104B. Distal end 104A may be coupled or affixed to distal tip 105.

A plurality of openings 202 are disposed within wall 108 of elongated member 104. Openings 202 may include cuts, voids, or any other discontinuity in wall 108 that modifies an ability of at least a portion of elongated member 104 to compress or expand in response to an external force applied on elongated member 104. In some examples, at least one opening of openings 202 extends from an outer surface of elongated member 104 to an inner surface of elongated member 104 defining an inner lumen of elongated member 104. In some examples, at least one opening of openings 202 extends partially from the outer surface of elongated member 104 towards the inner lumen of elongated member 104 without penetrating an inner surface of elongated member 104 defining the inner lumen.

In some examples, as illustrated in FIG. 2A, each of openings 202 extends along a reference plane orthogonal to longitudinal axis 106, e.g., such that openings 202 are visualized as a linear cut in the side view of elongated member 104. In some examples, one or more of openings 202 may define other shapes (e.g., curved, angular, arcs). In some examples, as illustrated in FIG. 2A, each of openings 202 define same dimensions as other openings 202 and/or openings 202 define a same density of openings 202 along the length of elongated member 104. In some examples, the dimensions of openings 202 and/or the density of openings 202 vary along the length of elongated member 104, e.g., to cause distal section 110A to exhibit increased flexibility relative to proximal section 110B of elongated member 104.

Elongated member 104 may define a locking channel 204. Locking channel 204 may be distal to openings 202 or between longitudinally adjacent openings 202. Locking channel 204 may be sized to receive a locking element 204. Locking element 204 may be configured to affix distal section 102A of core wire 102 to elongated member 104. Locking element 204 may be permanently or removably affixed to core wire 102 and/or to elongated member 104. When locking element 204 is affixed to core wire 102 and elongated member 104, locking element 204 may inhibit unintended movement of core wire 102 along longitudinal axis 106 relative to elongated member 104, or vice versa.

For example, the flexibility of distal section 110A may enable a clinician to form distal section 110A into a particular shape. However, when a clinician is shaping elongated member 104, the bending and/or tensile forces exerted on elongated member 104 may cause fracturing of elongated member 104, e.g., near one or more of openings 202. As one example, fractures may extend between openings 202 and cause partial or complete separation of portions of elongated member 104 form the remainder of elongated member 104. As another example, fractures may extend around the outer perimeter of elongated member 104 to cause complete separation of distal end 104A of elongated member 104 from proximal end 104B of elongated member 104.

Locking element 206 may enable guidewire 100 to retain separated portions of elongated member 104. Since locking element 206 is affixed to core wire 102 as well as to elongated member 104, locking element 206 may inhibit distal movement of separated portions of elongated member 104 relative to core wire 102 and past a distal end of core wire 102. In such examples, locking element 206 may retain separated portions of elongated member 104 and facilitate complete removal of the separated portions of elongated member 104 from the body of the patient. In contrast, another identical guidewire without locking element 206 may be unable to retain separated portions of elongated member 104, as the separated portions of elongated member 104 may be unrestricted from advancing distally along longitudinal axis 106 and past a distal end of core wire 102 to completely separate from the remainder of guidewire 100.

Locking element 206 may inhibit unintended movement of elongated member 104 relative to core wire 102 instead of or in addition to distal tip 105. In some examples, distal tip 105 may be affixed to both core wire 102 and to elongated member 104 (e.g., via distal end 104A of elongated member 104), and may assist in preventing unintended distal movement and/or separation of portions of elongated member 104 relative to core wire 102. However, distal tip 105 may partially or completely separate from one or more of core wire 102 or elongated member 104 during the shaping process for guidewire 100. In the event that distal tip 105 becomes separated from core wire 102 or elongated member 104 and/or become otherwise incapable of inhibiting movement between core wire 102 and elongated member 104, locking element 206 may inhibit unintended movement of elongated member 104 relative to core wire 102. In this way, locking element 206 may provide an additional point of safety to prevent unintended separation of portions of guidewire 100. Locking element 206 may define an additional point of failure (e.g., a third point of failure) required for complete separation of at least a portion of elongated member 104 from the rest of guidewire 100. The other points of failure may include a facture of elongated member 104 and a separation of distal tip 105 from core wire 102 and/or elongated member 104.

In some examples, if distal tip 105 separates from core wire 102, core wire 102 remains affixed to elongated member 104 via locking element 206. In such examples, locking element 206 remains affixed to core wire 102 and interfaces with portions of wall 108 defining locking channel 204 to inhibit movement of elongated member 104 relative to core wire 102 or vice versa. In some examples, distal tip 105 remains affixed to locking element 206 to inhibit complete separation of distal tip 105 from core wire 102 and elongated member 104 and facilitate retrieval of a partially separated distal tip 105 from within the patient.

FIG. 2B is a conceptual diagram illustrating a cross-sectional view of distal section 103A of the example guidewire 100 of FIG. 2A, the cross-section being taken along line A-A in FIG. 2A. As illustrated in FIG. 2B, guidewire 100 includes core wire 102, elongated member 104, support member 203, and distal tip 105. Elongated member 104 may include an outer surface 208 and an inner surface 210. Inner surface 210 may define an inner lumen 212. Support member 203 may be disposed within inner lumen 212 of elongated member 104 and around at least a portion of core wire 102. A distal tip 218 of a distal section 214 of core wire 102 may be connected to distal tip 105. Distal section 214 of core wire 102 may be disposed within inner lumen 206 and extend through an aperture 216 within locking element 206. Distal section 214 may define a reduced-diameter portion and/or a distal-most section of distal section 102A of core wire 102.

Distal section 110A and proximal section 110B may have a unitary body construction, e.g., may be formed as one body, such that a wall 108 of elongated member 104 is continuous along the entire length of elongated member 104, such that elongated member 104 is a single, seamless tubular body. A seamless elongated member 104 may, for example, be devoid of any seams (e.g., a seam formed from joining two separate tubular bodies together at an axial location along longitudinal axis 106), such that the seamless elongated member 104 is a unitary body, rather than multiple, discrete bodies that are separately formed and subsequently connected together. A seamless elongated member 104 may be easier to slide over another device, e.g., a guide member, compared to an elongated member formed from two or more longitudinal sections that are mechanically connected to each other because the seamless elongated member may define a smoother inner lumen. In contrast, joints between sections of an elongated member that are formed from two or more longitudinal sections may define surface protrusions or other irregularities along the inner lumen which may interfere with the passage of devices through the inner lumen. In addition, a seamless elongated member 104 may help distribute pushing and rotational forces along the length of guidewire 100. Thus, the seamless elongated member 104 may help contribute to the pushability of guidewire 100.

In some examples, a thickness of wall 108 of elongated member 104 is substantially constant along a length of elongated member 104. In other examples, the thickness of wall 108 varies along a length of elongated member 104. For example, the thickness of wall 108 may decrease toward distal end 104A (e.g., the thickness of wall 108 may decrease from proximal end 104B to distal end 104A of elongated member 104, or may decrease from a proximal end of distal section 110A to a distal end of distal section 110A). The thickness of linear wall 108 may linearly or non-linearly increase from distal end 104A. In some examples, the thickness of linear wall 108 may linearly or non-linearly decrease from proximal end 104B and/or from the distal end of distal section 110A. For example, the thickness of wall 108 may decrease from about 0.33 millimeters (mm) at the proximal end of elongated member 104 to about 0.0127 mm (about 0.0005 inches) at the distal end of elongated member 104. However, other wall thicknesses may be used in other examples, and may depend on the particular procedure for which guidewire 100 is used.

In some examples, elongated member 104 may define a same outer diameter along the length of elongated member 104 as the thickness of wall 108 varies along the length of elongated member 104. In such examples, an inner diameter of inner lumen 212 may vary along the length of elongated member 104, e.g., to maintain the same outer diameter. In some examples, inner lumen 212 may define a same inner diameter along the length of elongated member 104 as the thickness of wall 108 varies along the length of elongated member 104. In some examples, both the outer diameter of elongated member 104 and the inner diameter of inner lumen 212 may vary as the thickness of wall 108 varies along the length of elongated member 104.

In the example of FIG. 2B, openings 202 extend through the entire thickness of wall 108 (measured in a direction perpendicular to longitudinal axis 106 of guidewire 100, which may also be a longitudinal axis of elongated member 104). In other examples, openings 202 extend partially through wall 108. For example, openings 202 may extend from the outer surface of elongated member 104 towards but not reaching inner lumen 212 of elongated member 104. The depth of the partial opening 202 may be described as a percentage of the thickness of wall 108 or as a unit of length (e.g., millimeters). In some examples, the depth of a partial opening 202 may be about 20% to about 80% of the thickness of wall 108, such as about 50% to about 75% of the thickness of wall 108. In contrast, a through-opening 202 may extend through 100% of the thickness of wall 108.

Openings 202 may be formed on elongated member 104 using one or more techniques. In some examples, openings 202 may be etched, laser cut, or mechanically cut via a blade, router, abrasion disk, or the like into a tubular body or other material from which elongated member 104 is formed. In other examples, elongated member 104 may be formed by winding a ribbon of an elongated member material (e.g., PTFE) around a beading. As another example, elongated member 104 may be formed using an additive manufacturing process (also referred to as a three-dimensional printing technique in some examples). Openings 202 may then be defined during the additive manufacturing.

Support member 203 is configured to increase the structural integrity of guidewire 100 while allowing guidewire 100 to remain relatively flexible. For example, support member 203 may be configured to help guidewire 100 substantially maintain its cross-sectional shape or at least help prevent guidewire 100 from buckling or kinking as it is navigated through tortuous anatomy. In some examples, guidewire 100 may include another layer, such as a support layer, that adheres support member 203 to elongated member 104. Support member 203, together with elongated member 104, may help distribute both pushing and rotational forces along a length of guidewire 100, which may help prevent kinking of guidewire 100 upon rotation of guidewire 100 or help prevent buckling of guidewire 100 upon application of a pushing force to core wire 102. As a result, a clinician may apply pushing forces, rotational forces, or both, to proximal section 103B of guidewire 100, and such forces may cause distal section 103A of guidewire 100 to advance distally, rotate, or both, respectively. Support member 203 may define an inner lumen configured to retain distal section 214 of core wire 102.

In the example of FIG. 2B, support member 203 extends along only a portion of a length of core wire 102. For example, a proximal end of support member 203 may be positioned distal to proximal end 104B of elongated member 104. The outer diameter of core wire 102 may reduce from proximal section 103B to distal tip 218, e.g., to allow for placement of distal section 214 in inner lumen 212 of elongated member 104 and in the inner lumen of support member 203 without increasing the outer diameter of distal section 103A of guidewire 100.

In some examples, support member 203 includes a generally tubular braided structure, a coil member defining a plurality of turns, e.g., in the shape of a helix, or a combination of a braided structure and a coil member. Thus, although examples of the disclosure describe support member 203 as a coil, in some other examples, the catheter bodies described herein include a braided structure instead of a coil or a braided structure in addition to a coil. For example, a proximal section of support member 203 may include a braided structure and a distal section of structural support member 203 may include a coil member. Support member 203 can be made from any suitable material, such as, but not limited to, a metal (e.g., a nickel titanium alloy (Nitinol) or stainless steel), a polymer, a fiber, or any combination thereof.

Support member 203 may be coupled, adhered, or mechanically connected to at least a portion of an inner surface of elongated member 104, such as via a support layer. The support layer may be a thermoplastic material or a thermoset material, such as a thermoset polymer or a thermoset adhesive. In some cases, the material forming the support layer may have elastic properties, such that there may be a tendency for the support layer to return to a resting position. In some examples, the support layer is positioned between the entire length of support member 203 and elongated member 104. In other examples, the support layer is only positioned between a part of the length of support member 203 and elongated member 104.

Locking channel 204 may retain locking element 216 within elongated member 104. Locking channel 204 may extend from a first location (e.g., axial and/or circumferential position) along outer surface 208 of elongated member 104 towards a second location along outer surface 208 of elongated member 104. The second location may be directly opposite the first location along the outer perimeter of elongated member 104, e.g., such that the second location is separated from the first location by a distance equal to the outer diameter of elongated member 104. In some examples, as illustrated in FIG. 2B, locking channel 204 extends through elongated member 104 (e.g., through wall 108 of elongated member 104) from the first location to the second location. In some examples, locking channel 204 extends towards the second location but does not extend from outer surface 208 at the second location. The first location and the second location may define a same longitudinal position or may define different longitudinal positions relative to longitudinal axis 106.

Locking channel 204 may be defined by two or more locking recesses 222A, 222B (collectively referred to as “locking recesses 222”). Each of locking recesses 222 may extend at least partially through wall 108 at or near a respective location along outer surface 208. For example, first locking recess 222A extends from outer surface 208 at the first location along outer surface 208 to the inner surface 210 of elongated member 104, e.g., to define a through opening from outer surface 208 to inner surface 210. As another example, second locking recess 222B extends from inner surface 210 towards the second location along outer surface 208. In some examples, second locking recess 222B extends from inner surface 210 to outer surface 208 at second location, e.g., to define a through opening from outer surface 208 to inner surface 210. Each of locking recesses 222 may extend between outer surface 208 and inner surface 210 along a respective reference axis. Each respective reference axis may interest with longitudinal axis 106 and may extend along a radius of elongated member 104.

Each of locking recesses 222 may define a same or different geometry. In examples in which locking recesses 222 define a same geometry, locking recesses 222 define locking channel 204 such that locking element 206 may be inserted into locking channel 204 along multiple directions (e.g., from first locking recess 222A and into second locking recess 222B or vice versa). In some examples, locking recesses 222 may define different geometry such that locking element 206 may only be inserted into locking channel 204 in a single direction (e.g., from first locking recess 222A and into second locking recess 222B, but not vice versa.). Sidewalls of wall 108 defining locking recesses 222 may interface with surfaces on locking element 206 to inhibit excess travel of locking element 206 within locking channel 204 and to align an aperture 216 in locking element 206 with longitudinal axis 106. For example, sidewalls of wall 108 defining first locking recess 222A may interface with locking element 206 to inhibit further travel of locking element 206 into second locking recess 222B once aperture 216 aligns with longitudinal axis 106.

Locking recesses 222 may be separated from a proximal end of distal tip 105 of guidewire 100 by a distance 217. In such examples, each of locking recesses 222 may be defined by a plurality of sidewalls defined by wall 108 of elongated member 104. In some examples, locking channel 204 may be directly adjacent to the proximal end of distal tip 105. In such examples, each of locking recesses 222 may be at least partially defined by distal tip 105.

Locking element 206 may be configured to be inserted into locking channel 204. Once locking element 206 is inserted into locking channel 204, aperture 216 on locking element 206 may be aligned with longitudinal axis 106. Aperture 216 may define an opening extending from a proximal surface of locking element 206 to a distal surface of locking element 206. Aperture 216 may be size to retain at least a portion of distal section 214 of core wire 102. Aperture 216 may define a cross-section with a circular, triangular, rectangular, quadrilateral, pentagonal, hexagonal, or other geometric shape. Aperture 216 extend along an axis orthogonal to distal surface and/or proximal surface of locking element 206.

Once locking element 206 is positioned within locking channel 204, locking element 206 may be removably or permanently affixed to elongated member 104. In some examples, one or more surfaces of locking element 206 are affixed to respective portions of wall 108 defining sidewalls of one or more of locking recesses 222. Locking element 206 may be affixed to elongated member 104 via an adhesive, a mechanical fit, a solder material, a weld, or the like. Locking element 206 may be soldered to welded to elongated member 104, e.g., to increase tensile strength of bonds between locking element 206 and elongated member 104. Locking element 206 may be formed from one or more biocompatible materials including, but is not limited to, a platinum alloy.

Distal section 214 of core wire 102 may extend through aperture 216 of locking element 206. Distal section 214 may define a reduced cross-section relative to a more proximal section of core wire 102. Distal section 214 may define a cross section with a circular, oval, triangular, rectangular, quadrilateral, pentagonal, hexagonal or other geometric shapes. Distal section 214 of core wire 102 may include a fixation feature 220 configured to affix distal section 214 to locking element 206 or otherwise inhibit proximal movement of core wire 102 relative to locking element 206. In some examples, fixation feature 220 allows core wire 102 to move distally relative to locking element 206 but inhibits proximal movement of a portion of core wire 102 at, about, or distal to fixation feature 220 from retracting proximally from within aperture 216 of locking element 206. Fixation feature 220 may cause core wire 102 to define an increased cross-section at or about fixation feature 220. The increased cross-section may define a height or width greater than a heigh or a width of the aperture 216, e.g., to inhibit proximal movement of fixation feature 220 past aperture 216. Fixation feature 220 may include, but are not limited to, a solder material positioned on distal section 214 at a location distal to locking element 206, an adhesive or solder material configured to affix distal section 214 to locking element 206, or a section of core wire 102 defining an increased cross section (e.g., via a pinching or coining process).

Distal tip 105 may be affixed to distal end 104A of elongated member 104 and to distal tip 218 of core wire 102. In the event of fracture and/or separation of a portion of elongated member 104, distal tip 105 may affix core wire 102 to elongated member 104 to inhibit unintended distal movement of a separated portion of elongated member 104 past distal tip 218 of core wire 102 and completely separate from the remainder of guidewire 100. In the event that distal tip 105 separates from one or more of elongated member 104 or core wire 102, locking element 106 may affix core wire 102 to elongated member 104 to inhibit unintended movement between core wire 102 and elongated member 104. In some examples, where distal tip 105 is affixed to locking element 206, locking element 206 may further inhibit separation of distal tip 105 from the remainder of guidewire 100.

FIG. 2C is a conceptual diagram illustrating another example cross-sectional view of distal section 103A of the example guidewire 100 of FIG. 2A, the cross-section being taken along line B-B in FIG. 2A. As illustrated in FIG. 2C, locking element 206 is disposed within a locking channel 204 within elongated member 104. Locking channel 204 may be defined by locking recesses 222. Each locking recess 222 may define a same shape and/or same dimensions as another locking recess 222, as illustrated in FIG. 2C. In such examples, locking element 206 may be inserted in locking channel 204 via a plurality of different locking recesses 222 (e.g., via locking recess 222A or via locking recess 222B, as illustrated in FIG. 2C). In some examples, each locking recess 222 may define different shapes and/or dimensions as another locking recess 222. In such examples, locking element 206 may be inserted into locking channel 204 via a specific locking recess 222 of the plurality of locking recesses 222, e.g., via locking recess 222A only.

Each of locking recesses 222 may extend from inner surface 210 of elongated member 104 to outer surface 208 of elongated member 104, as illustrated in FIG. 2C, or may extend partially from inner surface 210 towards outer surface 208 (e.g., so as to define a blind locking recess extending from inner surface 210). Each of locking recesses 222 may be defined by one or more sidewalls 224. In some examples, as illustrated in FIG. 2C, each of locking recesses 222 is defined by four or more sidewalls 224. Each locking recess 222 may be defined by a different number of sidewalls 224 and/or sidewalls 224 of different dimensions than another of locking recesses 222.

In some examples, wherein one of locking recesses 222 (e.g., locking recess 222B) is a blind locking recess, the respective locking recess may retain one end (e.g., end 230A) of locking element 206 and facilitate alignment of aperture 216 within locking element 206 with distal section 214 of core wire 102 within inner lumen 212 of elongated member 104.

Locking element 206 may extend from a first end 230A to a second end 230B along a reference axis 215. Locking element 206 may define one or more outer surfaces 226. At least one outer surface 226 of locking element 206 may interface with a corresponding sidewall 224 to align aperture 216 with distal section 214 of core wire 102. In some examples, outer surface(s) 226 interface with sidewall(s) 224 to inhibit overtravel of locking element 206 within locking channel 204, e.g., in a direction along reference axis 215 and towards a far side of locking channel 204.

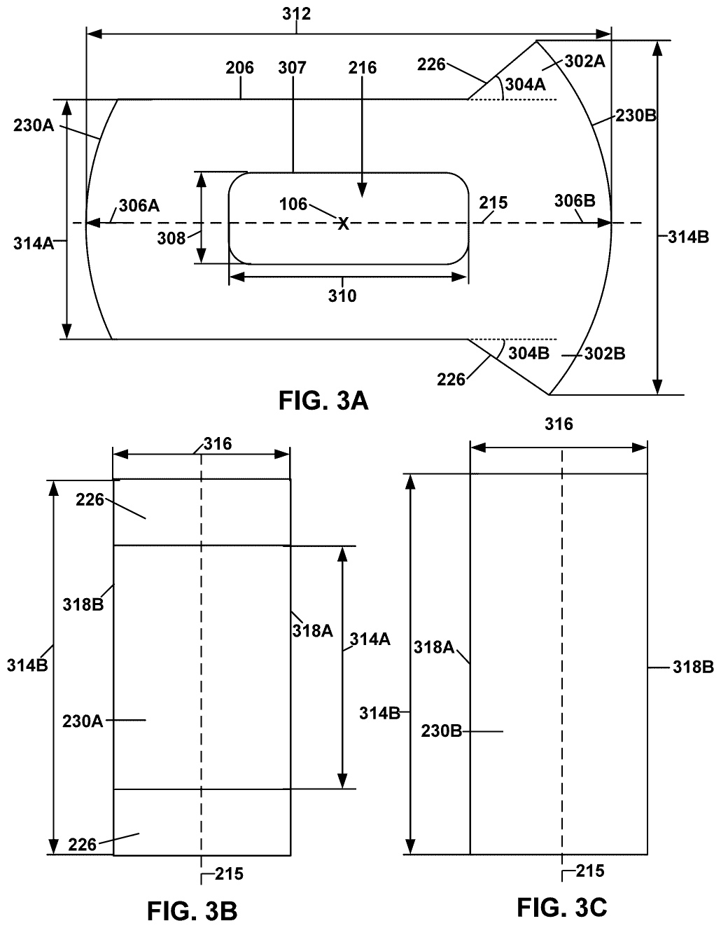

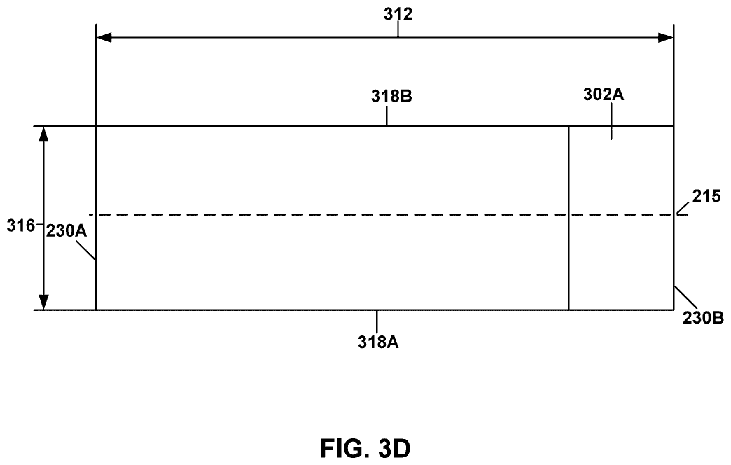

FIG. 3A is a conceptual diagram illustrating a front view of an example locking element 206. FIG. 3B is a conceptual diagram illustrating a side view of first end 230A of example locking element 206 of FIG. 3A. FIG. 3C is a conceptual diagram illustrating a side view of second end 230B of example locking element 206 of FIG. 3A. FIG. 3D is a conceptual diagram illustrating a top view of example locking element 206 of FIG. 3A.

As illustrated in FIGS. 3A-3D, locking element 206 may extend along reference axis 215 from a first end 230A to a second end 230B (collectively referred to herein as “ends 230”). Each end 230 of locking element 206 may define an angular, flat, or curved surface. In some examples, where ends 230 define curved surfaces, as illustrated in FIG. 3A, each end 230 may define a radius of curvature 306A, 306B (collectively referred to as “radii of curvature 306”). Radii of curvature 306 cross different ends 230 may be uniform or may be different. Radii of curvature 306 may be the same as the radius of curvature of elongated member 104, e.g., such that when locking element 206 is disposed within locking channel 204 in elongated member 104, ends 230 may define a smooth, continuous surface with elongated member 104.

Locking element 206 may define a length 312 along reference axis 215 from first end 230A to second end 230B. Length 312 may be a longest distance between first end 230A and second end 230B. Length 312 may be less than or equal to an outer diameter of elongated member 104, e.g., such that when locking element 206 is disposed within locking channel 204, locking element 206 does not increase an overall outer diameter of guidewire 100. Locking element 206 may extend along longitudinal axis 106 from front surface 318A to rear surface 318B. Locking element 206 may define a thickness 316 along longitudinal axis 106 from front surface 318A to rear surface 318B. Thickness 316 may be uniform across the entirety of locking element 206 or may vary at specific regions (e.g., around extensions 302A, 302B) of locking element 206.

Locking element 206 may include one or more extensions 302A, 302B (collectively referred to herein as “extensions 302”). Locking element 206 may include one extension 302, two extensions 302, or three or more extensions 302. Each extension 302 may extend from one of front surface 318A or rear 318B towards the other of front surface 318A or rear surface 318B. Each extension 302 may extend away from reference axis 215. In some examples, where one or more extensions define an outer surface 226 angled away from reference axis 215, as illustrated in FIG. 3A, each of extensions 302 may define an angle 304 defining the slope of the corresponding outer surface 226 relative to reference axis 215. Angles 304 of extensions 302 may be uniform or may be different.

Extensions 302 may be positioned around one of ends 230 of locking element 206. For example, as illustrated in FIGS. 3A-3D, extensions 302 are positioned around second end 230B of locking element 206. Extensions 302 may increase a width of locking element 206 relative to another end 230 of locking element 206 with no extensions 302. For example, as illustrated in FIG. 3A, first end 230A without any extensions 302 define a first width 314A along a reference axis orthogonal to reference axis 215 and second end 230B with extensions 302A, 302B define a second width 314B along the same reference axis, second width 314B being greater than first width 314A. In other examples, where extensions 302 are disposed around or adjacent to first end 230A, first width 314A may be greater than second width 314B. The difference in width between first width 314A and second width 314B may inhibit extensions 302 of locking element 206 from passing through a reduced-width portion of locking channel 204, thereby maintaining locking element 206 in proper alignment and position within locking channel 204.

Outer surfaces 226 of extensions 302 may interface with corresponding sidewalls 224 of locking channel 204 of elongated member 104 to properly position locking element 206 within locking channel 204 and relative to core wire 102, e.g., such that distal section 214 of core wire 102 may pass through aperture 216 of locking element 206. For example, outer surfaces 226 of extensions 302 may contact corresponding sidewalls 224 of elongated member 104 to inhibit excess travel of locking element 206 towards a far side of locking channel 204 once aperture 216 is aligned with core wire 102. In some examples, where extensions 302 are angled, corresponding sidewalls 224 may be angled at the same angles (e.g., at same angles 304 as respective extensions 302).

Locking element 206 may define aperture 216 extending through locking element 206 from front surface 318A to rear surface 318B. Front surface 318A may be parallel to rear surface 318B. When locking element 206 is disposed within locking channel 204 in elongated member 104, aperture 216 may align with longitudinal axis 106. Locking element 206 may define aperture 216 with circular, triangular, square, rectangular, quadrilateral, pentagonal, hexagonal, or other polygonal-shaped cross-sections. Locking element 206 may define an inner surface 307 defining aperture 216. Aperture 216 may define a same or different cross-sectional shape as distal section 214 of core wire 102. Aperture 216 may define a length 310 along reference axis 215 and a width 308 along a reference axis orthogonal to reference axis 215. Length 310 and width 308 of aperture 216 may be greater than or substantially the same as the corresponding measurements on distal section 214 of core wire 102, e.g., to facilitate insertion of distal section 214 of core wire 102 through aperture 216. In some examples, wherein distal section 214 of core wire 102 is configured to be affixed to locking element 206 via a pinching or coining process, length 310 and width 308 may be less than the corresponding measurements on distal section 214 of core wire 102.

Inner surface 307 of locking element 206 may define an outer perimeter of aperture 216. Inner surface 307 may be configured to interface with an outer surface of core wire 102 when distal section 214 of core wire 102 is disposed within aperture 216. Locking element 206 may transmit torque from core wire 102 to elongated member 104 via a first interface between core wire 102 and inner surface 307 of locking element 206 and via a second interface between one or more outer surfaces 226 of locking element 206 and one or more sidewalls 224 of elongated member 104.

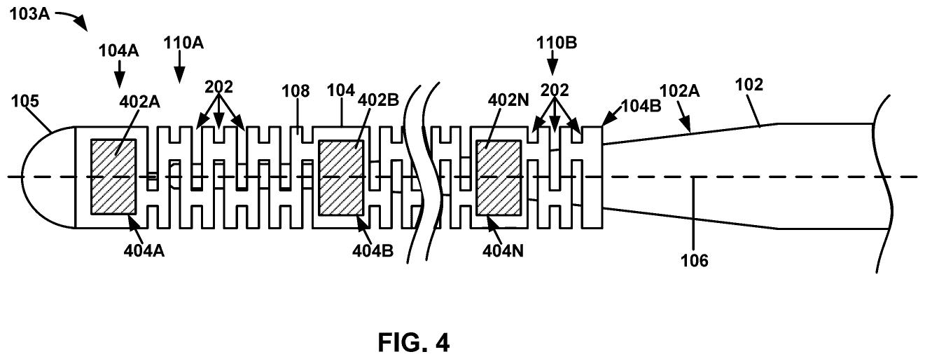

FIG. 4 is a conceptual diagram illustrating another example of elongated member 104 of guidewire 100 of FIG. 1 with a plurality of example locking elements 402A-N (collectively referred to herein as “locking elements 402”). Elongated member 104 may include two, three, or four or more locking elements 402. Each of locking elements 402 may be configured to be inserted into any of locking channels 404A-N (collectively referred to herein as “locking channels 404”) or into a specific corresponding locking channel 404 of locking channels 404.

Locking elements 402 may be configured to affix core wire 102 to elongated member 104. Each locking element 402 may be configured to be affixed to elongated member 104 in accordance with one or more of the examples previously described herein. Each locking element 402 may be configured to interact with one or more of core wire 102 or elongated member 104 in accordance with one or more of the examples previously described herein. Locking elements 402 may define uniform shapes and/or dimensions or may define different shapes and/or dimensions. Locking element 402A may define a same shape and/or dimensions as locking element 206. Locking elements 402B-N may define different shapes and/or dimensions as locking element 402A.

In some examples, instead of one or more locking elements 402, one or more inserts may be inserted into one or more of locking channels 404. The one or more inserts may be configured to seal the one or more locking channels 404 in place of a corresponding locking element 402. The one or more inserts may not be coupled or affixed to core wire 102 and may increase tensile strength and/or tensile stiffness of elongated member 104 compared to an identical elongated member 104 with one or more empty locking channels 404 (i.e., without corresponding locking elements 402 in the one or more locking channels 404).

Locking channels 404 may be disposed at different locations along elongated body 104. For example, as illustrated in FIG. 4, locking channels 404 may be disposed at different longitudinal positions between distal end 104A and proximal end 104B and along longitudinal axis 106. One or more of locking elements 402 may be affixed to elongated member 104 and/or core wire 102, e.g., in a same manner as described above with respect to locking element 206. As compared to a single locking element 206 (e.g., as illustrated in FIGS. 2A-2B), disposing two or more locking elements 402 within elongated member 104 may increase torque transmission between core wire 102 and elongated member 104 and/or provide additional points of safety before unintentional separation of portions of elongated body 104 from core wire 102 and/or distal tip 105 of guidewire 100. For example, in an instance of fracture around a proximal section 110B of elongated member 104, each of one or more locking elements 402 distal to the fracture and distal tip 105 would need to separate from core wire 102 before a portion of elongated member 104 distal to the fracture separates from the remainder of guidewire 100.

Locking channels 404 may extend at least partially through elongated member 104 along a same reference axis and/or same reference plane or along different reference axes and/or different reference planes. For example, locking channel 404A may extend along a first reference axis orthogonal to longitudinal axis 106 and locking channel 404B may extend along a second reference axis orthogonal to longitudinal axis 106 and different from the first reference axis (e.g., orthogonal to the first reference axis). Different reference axes and/or different reference planes for different locking channels 404 may inhibit bias in flexure of elongated member 104 and allow for unbiased flexure of elongated member 104 in any direction away from longitudinal axis 106.

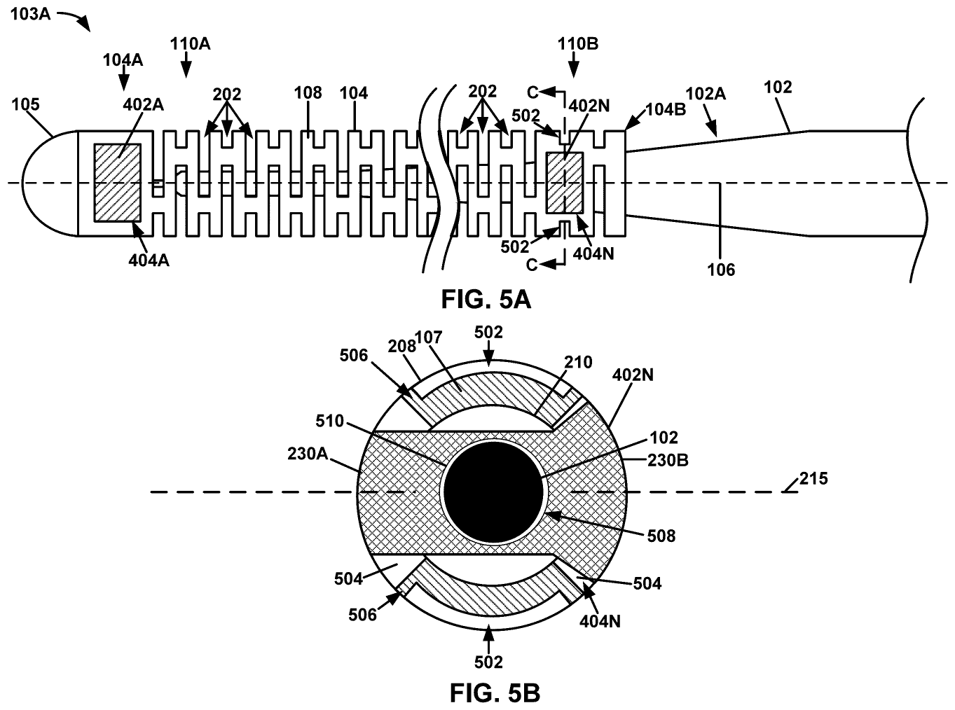

FIG. 5A is a conceptual diagram illustrating another example of elongated member 104 of guidewire 100 of FIG. 1 with a plurality of locking elements 402. FIG. 5B is a conceptual diagram illustrating a cross-sectional view of distal section 103A of example guidewire 100 of FIG. 5A, the cross-section being taken along line C-C in FIG. 5A. As illustrated in FIGS. 5A and 5B, one or more locking channels 404 (e.g., locking channel 404N) may be disposed longitudinally between and/or adjacent to openings 202 along elongated member 104. Elongated member 104 may include one or more openings 502 disposed partially along the outer perimeter of elongated member 104 and at overlapping longitudinal positions with one or more of locking channels 404 relative to longitudinal axis 106. Placement of openings 502 and locking channels 404 at same longitudinal positions may increase flexibility of elongated member 104 as compared to an identical elongated member 104 without any openings in the same longitudinal positions as locking channels 404.

Openings 502 may define the same shapes and/or dimensions as or different shapes and/or dimensions as one or more of openings 202. For example, as illustrated in FIG. 5B, openings 502 may extend from outer surface 208 towards outer surface 210 for a distance different from (e.g., less than) openings 202. In some examples, openings 502 extend around the outer perimeter of elongated body 104 for a reduced arc length compared to openings 202. The reduced depth and arc length of openings 502 may allow openings 502 to increase flexibility of elongated member 104 about a local region on elongated member 104 (e.g., as compared to an identical elongated member 104 with no openings 502) without reducing the tensile strength, torqueability, and/or structural integrity of elongated member 104 (e.g., as compared to an identical elongated member 104 with openings 502 having same dimensions as openings 202).

Elongated member 104 may include openings 502 at longitudinally overlapping locations with locking channels 404 along the entire longitudinal length of elongated member 104 or only within a localized region of elongated member 104 (e.g., within proximal section 110B only). In some examples, openings 202 closer to distal end 104A of elongated member 104 (e.g., within distal section 110A) define an increased density of openings 202 relative to openings 202 closer to proximal end 104B of elongated member 104. In such examples, openings 502 may only be disposed along proximal section 110B due to the increased flexibility provided by the increased density of openings 202 within distal section 110A and/or due to an increased reduction in structural integrity of distal section 110A of elongated member 104 resulting from placement of openings 502 within distal section 104A (e.g., due to a decrease in the width of sections of wall 107 of elongated member 104 between longitudinally adjacent openings 202.

As illustrated in FIG. 5B, openings 502 and locking recesses 504 of one or more locking channels 404 (e.g., locking channel 404N) may define open sections of elongated member 107 at the cross-section for the one or more locking channels 404. Locking recesses 504 may define same or similar shapes and dimensions as locking recesses 222 previously described herein. Uncut sections 506 of wall 107 of elongated member 104 may separate openings 502 and locking recesses 504. Uncut sections 506 may maintain elongated member 104 as a single, continuous component. A width of each of uncut sections 506 may be the same as or different from a width of uncut sections between longitudinally overlapping and circumferentially adjacent openings 202. In some examples, the width of uncut sections 506 are greater than the width between circumferentially adjacent openings 202, e.g., to maintain integrity of elongated member 104 and/or and inhibit fracture of elongated member 104 around openings 502 and locking recesses 504.

Locking element 402N may extend from first end 230A to second end 230B along reference axis 215. Locking element 402N may define different shapes and/or different dimensions (e.g., reduced widths 312, reduced thicknesses 316) compared to other locking elements 402 (e.g., more distal locking elements 402, locking element 404A, locking element 206). The different shapes and/or dimensions of locking element 402N may be disposed within and interface with a locking channel 404N of different shapes and/or dimensions as other locking channels 404 (e.g., more distal locking channels 404, locking channel 404A, locking channel 204). The difference in shape and/or dimensions of locking channel 404N may increase the flexibility, strength, or integrity of elongated member 104 around locking channel 404N relative to an identical locking channel 404N with a same shape and/or dimensions as other, more distal locking channels 404.

Locking element 402N may define an aperture 508 with different shape and/or dimensions as an aperture of a more distal locking element 402 (e.g., aperture 216 of locking element 206). Aperture 508 may be sized to receive a portion of core wire 102 proximal to distal section 214 of core ire 102, which may define a larger profile and/or a different shape than distal section 214. Inner surface 510 of locking element 402N defines aperture 508. Inner surface 510 may interface with an outer surface of core wire 102 to transmit torque from core wire 102 to a section of elongated member 104 around locking channel 404N.

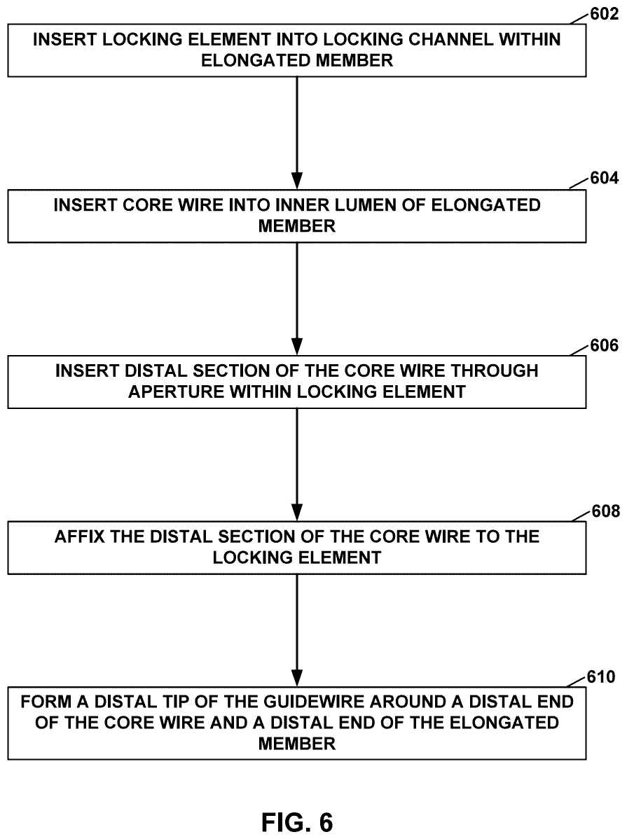

FIG. 6 is a flow diagram illustrating an example method of manufacturing the guidewire 100 of FIG. 1. While the example method of FIG. 6 is described herein primarily with reference to guidewire 100 of FIGS. 1-3D, the example method may be applied to any example guidewire 100 described in this disclosure.

A manufacturing system may insert locking element 206 into locking channel 204 within elongated member 104 (602). Elongated member 104 may extend from distal end 104A to proximal end 104B along longitudinal axis 106 and may define a distal section 110A and a proximal section 110B. An inner surface 210 of elongated member 210 may define an inner lumen 212 extending from distal end 104A to proximal end 104B. Elongated member 104 may include one or more openings 202 disposed over at least a portion of elongated member 104. Each opening 202 may extend from outer surface 208 of elongated member 104 towards inner surface 210 of elongated member 104. In some examples, one or more openings 202 extend from outer surface 208 through to inner surface 210. Longitudinally adjacent and circumferentially adjacent openings 202 may be separated by uncut sections of elongated member 104.

Elongated member 104 may include a locking channel 204 disposed along distal section 110A. Locking channel 204 may be disposed at a location within distal section 110A and distal to openings 202. Locking channel 204 may be defined by one or more locking recesses 222. Each of locking recesses 222 may be defined by one or more sidewalls 224. One of locking recesses 222 may be a blind recess (i.e., may extend from inner surface 210 towards but not through to outer surface 208). Locking element 206 may be inserted into locking channel 204 via one or more of locking recesses 222.

When locking element 206 is disposed with locking channel 204, outer surfaces 226 of extensions 302 on one end of locking element 206 may interface with sidewalls 224 of elongated member 104 to align an aperture 216 of locking element 206 within inner lumen 212 (e.g., with longitudinal axis 106). In some examples, where one of locking recesses 222 is a blind recess, the one locking recess 222 inhibits movement of locking element 206 out of a far side of locking channel 204 (e.g., opposite a side of entry of locking element 206 into locking channel 204) and aligns aperture 216 within inner lumen 212 of elongated member 104.

In some examples, after locking element 206 is disposed within locking channel 204, the manufacturing system may affix (removably or permanently) locking element 206 to elongated member 104. The manufacturing system may affix one or more outer surfaces 226 of locking element 206 to one or more corresponding sidewalls 224 of elongated member 104. The manufacturing system may affix locking element 206 to elongated member 104 via an adhesive, a mechanical fit, a solder material, a weld, or the like.

The manufacturing system may insert core wire 102 into inner lumen 212 of elongated member 104 (604). The manufacturing system may insert a distal tip 218 of core wire 102 into inner lumen 212 of elongated member 104 via proximal end 104B of elongated member 104. The manufacturing system may advance distal tip 218 of core wire 102 towards distal end 104A of elongated member 104 along longitudinal axis 106 and within inner lumen 212. When the manufacturing system advances distal tip 218 of core wire 102 within inner lumen 212, the manufacturing system may advance core wire 102 in line with longitudinal axis 106.

The manufacturing system may insert distal section 214 of core wire 102 through aperture 216 within locking element 206 (606). Distal section 214 may define a reduced cross-sectional area relative to a more proximal portion of core wire 106. The cross-sectional area of distal section 214 may be the same as or different from a cross-sectional area of aperture 216 of locking element 206. The manufacturing system may advance distal tip 218 of core wire 102 distally through aperture 216. The manufacturing system may continue to advance distal tip 218 of core wire 102 until distal tip 218 is at a same longitudinal position or is distal to distal end 104A of elongated member 104. When distal tip 218 of core wire 102 is at or distal to distal end 104A of elongated member 104, distal section 214 of core wire 102 may extend through aperture 216. One or more outer surfaces along distal section 214 of core wire 102 may interface with an inner surface 307 of locking element 206. Distal section 214 of core wire 102 may transmit torque to locking element 206 via the interface with inner surface 307.

The manufacturing system may affix distal section 214 of core wire 102 to locking element 206 (608). The manufacturing system may removably or permanently affix distal section 214 of core wire 102 via fixation feature 220. In some examples, the manufacturing system forms fixation feature 220 along distal section 214 of core wire 102 and distal to locking element 206 to inhibit unintended distal movement of locking element 206. When locking element 206 is affixed to core wire 102 and/or inhibited from moving distally relative to core wire 102 via fixation feature 220, locking element 206 may inhibit unintended distal movement of at least a portion of elongated member 104, thereby retaining separated portions of elongated member 104 (e.g., due to fractures within elongated member 104) with core wire 102. Locking element 206 may be affixed to core wire 102 via a solder material, an adhesive, a weld, a mechanical fit, or a pinching or coining process.

The manufacturing system may form distal tip 105 of guidewire 100 around distal end 218 of core wire 102 and distal end 104A of elongated member 104 (610). The manufacturing system may shape a biocompatible material (e.g., a biocompatible glue, a biocompatible polymer) into a desired shape. The desired shape may be a blunted, semi-spherical, or otherwise atraumatic shape, e.g., to prevent unintended puncture of the tissue of the patient by distal tip 105. The manufacturing system may form distal tip 105 around or over distal tip 218 of core wire 102 and distal end 104A of elongated member 104. In some examples, the manufacturing system may form distal tip 102 over at least a portion of a front surface 318A of locking element 206, e.g., to further affix locking element 206 to core wire 102 and distal section 104A of elongated member 104. Distal tip 105 may couple distal section 110A of elongated member 104 to core wire 102 and inhibit unintended movement of distal section 110A relative to core wire 102. Distal tip 105 and locking element 206 may provide additional points of safety in additional to the structural integrity of elongated member 104 to prevent unintended separation of portions of guidewire 100.

The examples described herein may be combined in any permutation or combination. The disclosure herein describes all of the following examples.

Example 1: a guidewire comprising: a core wire defining a longitudinal axis; an elongated member extending along the longitudinal axis, the elongated member defining: an inner surface defining an inner lumen, wherein a distal section of the core wire is positioned within the inner lumen, a plurality of openings disposed along a portion of the elongated member, each opening of the plurality of openings extending from an outer surface of the elongated member towards the inner surface of the elongated member, a first locking recess distal to the plurality of openings, the first locking recess extending through the elongated member from a first location on the outer surface of the elongated member to the inner lumen, a second locking recess distal to the plurality of openings, the second locking recess extending from the inner surface of the elongated member towards a second location on the outer surface of the elongated member, wherein the second location is different from the first location, and wherein the first locking recess and the second locking recess defines a locking channel extending from the first location; and a locking element disposed within the locking channel and the inner lumen and affixed to the elongated member, the locking element defining an aperture, wherein a distal end of the core wire extends through the aperture, wherein the locking element is configured to inhibit unintended proximal movement of the core wire along the longitudinal axis relative to the elongated member.

Example 2: the guidewire of example 1, wherein the second locking recess extends through the elongated member from the inner surface of the elongated member to the second location.

Example 3: the guidewire of any of examples 1 and 2, wherein the first locking recess is defined by a plurality of sidewalls, wherein one or more sidewalls of the plurality of sidewalls is configured to interface with a corresponding surface of the locking element.

Example 4: the guidewire of example 3, wherein the plurality of sidewalls comprises a first plurality of sidewalls, two sidewalls of the first plurality of sidewalls defining a first width along a reference plane coincidental to the longitudinal axis, wherein the second locking recess is defined by a second plurality of sidewalls, two sidewalls of the second plurality of sidewalls defining a second width along the reference plane, and wherein the first width is greater than the second width.

Example 5: the guidewire of any of examples 3 and 4, wherein the locking element defines: a central body extending along a central axis of the locking element from a first end to a second end; and one or more extensions disposed at the second end of the central body and extending away from the central axis, wherein the central body defines a first width along a reference plane orthogonal to the central axis at the first end, wherein the one or more extensions define a second width along the reference plane, and wherein the second width is greater than the first width.

Example 6: the guidewire of example 5, wherein the one or more extensions of the one or more extensions are configured to interface with the first locking recess to retain the locking element within the locking channel.

Example 7: the guidewire of any of examples 3-6, wherein the locking element is configured to transfer a torque from the core wire to the elongated member via the interface between the one or more sidewalls and the corresponding surface of the locking element.

Example 8: the guidewire of any of examples 1-7, wherein when the locking element is disposed within the locking recess, and wherein the longitudinal axis extends through the aperture within the locking element.

Example 9: the guidewire of any of examples 1-8, wherein the core wire defines a fixation feature disposed at a location along the core wire distal to the locking element, and wherein to inhibit unintended proximal movement of the core wire along the longitudinal axis relative to the elongated member, the locking element is configured to interface with the fixation feature on the core wire to inhibit proximal movement of the distal end of the core wire through the aperture of the locking element.

Example 10: the guidewire of example 9, wherein the fixation feature and the aperture of the locking element each defines a respective width along a reference plane orthogonal to the longitudinal axis and along a major axis of the aperture, wherein the fixation feature and the aperture each defines a respective height along the reference plane and along a minor axis of the aperture, and wherein the fixation feature defines one or more of: a width of the fixation feature greater than a width of the aperture; or a height of the fixation feature greater than a height of the aperture.

Example 11: the guidewire of any of examples 9 and 10, wherein the fixation feature comprises a fixation material affixing the core wire to the locking element, wherein the fixation material comprises one or more of: an adhesive; or a solder material.

Example 12: the guidewire of any of examples 9-11, wherein the fixation feature is configured to be formed from one or more of a crimp or a weld at or proximal to the location along the core wire.

Example 13: the guidewire of any of examples 1-12, wherein the locking recess comprises a first locking recess and wherein the locking element comprises a first locking element, wherein the elongated member further comprises one or more second locking recesses disposed between longitudinally adjacent openings of the plurality of openings, wherein the guidewire further comprises one or more second locking elements, each second locking element being disposed within a second locking recess of the one or more second locking recesses, wherein each second locking element defines a second aperture and is affixed to the elongated member, wherein the distal portion of the core wire extends through the one or more second apertures of the one or more second locking elements, and wherein each second locking element inhibits the unintended distal movement of the elongated member and the unintended proximal movement the core along the longitudinal axis.

Example 14: the guidewire of any of examples 1-13, wherein the plurality of openings comprises: one or more first openings at different longitudinal locations than the locking recess along the elongated member; and one or more second openings disposed at a same longitudinal location as the locking recess and at different circumferential locations than the locking recess, wherein the one or more first openings and the one or more second openings are configured to facilitate flexure of the guidewire about a plane orthogonal to the longitudinal axis.

Example 15: the guidewire of any of examples 1-14, wherein the aperture defines a rectangular aperture, and wherein the core wire defines a rectangular cross-section, wherein the rectangular cross-section defines a cross wire width and a cross wire height along a reference plane orthogonal to the longitudinal axis, wherein the rectangular aperture defines an aperture width and an aperture height along the reference plane, and wherein the rectangular aperture and the rectangular cross-section define a same major axis and a same minor axis.

Example 16: the guidewire of example 15, wherein the core wire defines a first plurality of surfaces defining the rectangular cross-section, wherein the aperture defines a second plurality of surfaces defining the rectangular aperture, and wherein a first surface of the first plurality of surfaces is configured to interface with a second surface of the second plurality of surfaces to transfer a torque from the core wire to the locking element.

Example 17: the guidewire of any of examples 1-16, further comprising a distal tip affixed to a distal end of the elongated member and to the distal end of the core wire, wherein the distal tip is configured to inhibit unintended movement of the elongated member relative to the core wire.

Example 18: the guidewire of example 17, wherein the distal tip comprises a glue dome.

Example 19: the guidewire of any of examples 17 and 18, wherein the distal tip is affixed to the locking element, wherein the distal tip is configured to inhibit unintended movement between the elongated member, the core wire, and the locking element.

Example 20: a method of manufacturing a guidewire of claim 1, the method comprising: inserting the locking element into the locking channel on the elongated member via the first locking recess; inserting a distal end of the core wire through the inner lumen of the elongated member and the aperture of the locking element; affixing the distal end of the core wire to the locking element, wherein when the distal end of the core wire is affixed to the locking element, the locking element inhibits unintended proximal movement of the distal end of the core wire along the longitudinal axis relative to the elongated member; and forming a distal tip of the guidewire around the distal end of the core wire and a distal end of the elongated member, wherein the distal tip of the guidewire is configured to inhibit unintended movement of the core wire relative to the elongated member.

Example 21: the method of example 20, wherein inserting the locking element into the locking channel comprises: inserting the locking element into the locking channel until the one or more surfaces of the locking element interface with one or more sidewalls of the elongated member defining the first locking recess to inhibit unintended movement of the locking element along through the locking channel and towards the second locking recess.

Example 22: the method of example 21, wherein when the one or more surface of the locking element interfaces with the one or more sidewalls of the elongated member, the aperture of the locking element is positioned within the inner lumen of the elongated member such that the longitudinal axis of the elongated member extends through the aperture.

Example 23: the method of any of examples 20-22, wherein affixing the distal end of the core wire to the locking element comprises: forming a fixation feature on the core wire at a location distal to the locking element, wherein the locking element is configured to interface with the fixation feature to inhibit proximal movement of the distal end of the core wire through the aperture of the locking element.

Example 24: the method of example 23, wherein forming the fixation feature on the core wire at the location comprises one or more of: crimping the core wire at or proximal to the location, welding the core wire at the location, or applying a fixation material at the location, wherein the fixation material comprises one or more of: an adhesive, or a solder material.

Example 25: a locking element configured to be inserted into a locking channel of a guidewire, the locking element comprising: a central body extending along a first axis from a proximal surface to a distal surface and from a first end to a second end along a second axis, wherein the second axis is orthogonal to the first axis; one or more extensions extending away from the second end of the central body and away from the second axis, wherein each extension of the one or more extensions extend along the first axis from one of the distal surface or the proximal surface towards the other of the distal surface or the proximal surface; and an aperture extending along the first axis from the proximal surface to the distal surface, wherein the aperture is configured to receive a core wire of the guidewire when the locking element is inserted into the locking channel within an elongated body of the guidewire, and wherein the distal surface is configured to interface with the core wire to inhibit unintended proximal movement of the core wire along the first axis.

Example 26: the locking element of example 25, wherein the central body defines a first height of the central body along a third axis at the first end, wherein the third axis is orthogonal to the first axis and the second axis, wherein the one or more extensions define a second height of the central body along the third axis at the second end, and wherein the second height is greater than the first height.

Example 27: the locking element of any of examples 25 and 26, wherein the first end of the central body defines a first continuous curved surface defining a first radius of curvature along a reference plane orthogonal to the first axis, and wherein the second end defines a second continuous curved surface defining a second radius of curvature along the reference plane.

Example 28: the locking element of example 27, wherein the first radius of curvature is equal to the second radius of curvature, and wherein the first radius of curvature is equal to a radius of curvature of an outer perimeter of the elongated member of the guidewire.

Example 29: the locking element of any of examples 27 and 28, wherein the first end defines a first arc length between ends of the first continuous curved surface, wherein the second end defines a second arc length between ends of the second continuous curved surface, and wherein the second arc length is greater than the first arc length.

Example 30: the locking element of any of examples 25-29, wherein the locking element defines a width along the second axis from the first end to the second end, and wherein the width of the locking element is equal to a diameter of the elongated member of the guidewire.

Example 31: the locking element of any of examples 25-30, wherein the aperture comprises a rectangular aperture.

Example 32: the locking element of example 31, wherein the rectangular aperture defines a major axis and a minor axis, wherein the major axis extends along the second axis, and wherein the minor axis is orthogonal to the first axis and to the second axis.

Example 33: the locking element of any of examples 25-32, wherein the locking element defines reflectional symmetry across a reference plane extending along the second axis.

Example 34: the locking element of any of examples 25-33, wherein when the locking element is inserted into the locking channel of the guidewire, the first axis is parallel to a longitudinal axis of the guidewire and the second axis is orthogonal to the longitudinal axis.

Example 35: the locking element of any of examples 25-34, wherein the locking element comprises a platinum alloy.

Example 36: the locking element of any of examples 25-35, wherein the aperture comprises an circular aperture.

Various aspects of the disclosure have been described. These and other aspects are within the scope of the following claims.

What is claimed is:

1. A guidewire comprising:
a core wire defining a longitudinal axis;
an elongated member extending along the longitudinal axis, the elongated member defining:
an inner surface defining an inner lumen, wherein a distal section of the core wire is positioned within the inner lumen,
a plurality of openings disposed along a portion of the elongated member, each opening of the plurality of openings extending from an outer surface of the elongated member towards the inner surface of the elongated member,
a first locking recess distal to the plurality of openings, the first locking recess extending through the elongated member from a first location on the outer surface of the elongated member to the inner lumen,
a second locking recess distal to the plurality of openings, the second locking recess extending from the inner surface of the elongated member towards a second location on the outer surface of the elongated member, wherein the second location is different from the first location, and wherein the first locking recess and the second locking recess defines a locking channel extending from the first location; and
a locking element disposed within the locking channel and the inner lumen and affixed to the elongated member, the locking element defining an aperture, wherein a distal end of the core wire extends through the aperture,
wherein the locking element is configured to inhibit unintended proximal movement of the core wire along the longitudinal axis relative to the elongated member.

2. The guidewire of claim 1, wherein the second locking recess extends through the elongated member from the inner surface of the elongated member to the second location.

3. The guidewire of claim 2, wherein the first locking recess is defined by a plurality of sidewalls, wherein one or more sidewalls of the plurality of sidewalls is configured to interface with a corresponding surface of the locking element.

4. The guidewire of claim 3,
wherein the plurality of sidewalls comprises a first plurality of sidewalls, two sidewalls of the first plurality of sidewalls defining a first width along a reference plane coincidental to the longitudinal axis,
wherein the second locking recess is defined by a second plurality of sidewalls, two sidewalls of the second plurality of sidewalls defining a second width along the reference plane, and
wherein the first width is greater than the second width.

5. The guidewire of any of claim 4, wherein the locking element defines:
a central body extending along a central axis of the locking element from a first end to a second end; and
one or more extensions disposed at the second end of the central body and extending away from the central axis,
wherein the central body defines a first width along a reference plane orthogonal to the central axis at the first end,
wherein the one or more extensions define a second width along the reference plane, and
wherein the second width is greater than the first width.

6. The guidewire of claim 5, wherein the one or more extensions of the one or more extensions are configured to interface with the first locking recess to retain the locking element within the locking channel.

7. The guidewire of claim 6, wherein the locking element is configured to transfer a torque from the core wire to the elongated member via the interface between the one or more sidewalls and the corresponding surface of the locking element.

8. The guidewire of claim 7, wherein when the locking element is disposed within the locking recess, and wherein the longitudinal axis extends through the aperture within the locking element.

9. The guidewire of claim 8, wherein the core wire defines a fixation feature disposed at a location along the core wire distal to the locking element, and wherein to inhibit unintended proximal movement of the core wire along the longitudinal axis relative to the elongated member, the locking element is configured to interface with the fixation feature on the core wire to inhibit proximal movement of the distal end of the core wire through the aperture of the locking element.

10. The guidewire of claim 9,
wherein the fixation feature and the aperture of the locking element each defines a respective width along a reference plane orthogonal to the longitudinal axis and along a major axis of the aperture,
wherein the fixation feature and the aperture each defines a respective height along the reference plane and along a minor axis of the aperture, and
wherein the fixation feature defines one or more of:
a width of the fixation feature greater than a width of the aperture; or
a height of the fixation feature greater than a height of the aperture.

11. The guidewire of claim 10, wherein the fixation feature comprises a fixation material affixing the core wire to the locking element, wherein the fixation material comprises one or more of:
an adhesive; or
a solder material.

12. The guidewire of claim 11, wherein the fixation feature is configured to be formed from one or more of a crimp or a weld at or proximal to the location along the core wire.

13. The guidewire of claim 1, wherein the plurality of openings comprises:
one or more first openings at different longitudinal locations than the locking recess along the elongated member; and
one or more second openings disposed at a same longitudinal location as the locking recess and at different circumferential locations than the locking recess,
wherein the one or more first openings and the one or more second openings are configured to facilitate flexure of the guidewire about a plane orthogonal to the longitudinal axis.

14. The guidewire of claim 1,
wherein the aperture defines a rectangular aperture, and wherein the core wire defines a rectangular cross-section,
wherein the rectangular cross-section defines a cross wire width and a cross wire height along a reference plane orthogonal to the longitudinal axis,
wherein the rectangular aperture defines an aperture width and an aperture height along the reference plane, and
wherein the rectangular aperture and the rectangular cross-section define a same major axis and a same minor axis.

15. The guidewire of claim 14,
wherein the core wire defines a first plurality of surfaces defining the rectangular cross-section,
wherein the aperture defines a second plurality of surfaces defining the rectangular aperture, and
wherein a first surface of the first plurality of surfaces is configured to interface with a second surface of the second plurality of surfaces to transfer a torque from the core wire to the locking element.

16. The guidewire of claim 1, further comprising a distal tip affixed to a distal end of the elongated member and to the distal end of the core wire, wherein the distal tip is configured to inhibit unintended movement of the elongated member relative to the core wire.

17. The guidewire of claim 16, wherein the distal tip is affixed to the locking element, wherein the distal tip is configured to inhibit unintended movement between the elongated member, the core wire, and the locking element.

18. A method of manufacturing a guidewire of claim 1, the method comprising:
inserting the locking element into the locking channel on the elongated member via the first locking recess;
inserting a distal end of the core wire through the inner lumen of the elongated member and the aperture of the locking element;
affixing the distal end of the core wire to the locking element, wherein when the distal end of the core wire is affixed to the locking element, the locking element inhibits unintended proximal movement of the distal end of the core wire along the longitudinal axis relative to the elongated member; and
forming a distal tip of the guidewire around the distal end of the core wire and a distal end of the elongated member, wherein the distal tip of the guidewire is configured to inhibit unintended movement of the core wire relative to the elongated member.

19. The method of claim 18, wherein inserting the locking element into the locking channel comprises:
inserting the locking element into the locking channel until the one or more surfaces of the locking element interface with one or more sidewalls of the elongated member defining the first locking recess to inhibit unintended movement of the locking element along through the locking channel and towards the second locking recess.

20. The method of claim 19, wherein when the one or more surface of the locking element interfaces with the one or more sidewalls of the elongated member, the aperture of the locking element is positioned within the inner lumen of the elongated member such that the longitudinal axis of the elongated member extends through the aperture.
